(19) United States
(12) Patent Application Publication
(10) Pub. No.: US 2021/0185960 A1
(43) Pub. Date: Jun. 24, 2021

(54) SAFFLOWER VARIETY SXT BRIGHT CITRINE

(71) Applicant: Sensient Colors, LLC, St. Louis, MO (US)

(72) Inventors: Stefan ALLINGER, Friedersbach (AT); Rudolf MARCHANT, Friedersbach (AT); Vergel C. CONCIBIDO, Maryland Heights, MO (US); Jörg MEYER, St. Charles, MO (US)

(21) Appl. No.: 17/102,096

(22) Filed: Nov. 23, 2020

Related U.S. Application Data
(60) Provisional application No. 62/939,033, filed on Nov. 22, 2019.

(51) Int. Cl. A01H 6/14; A01H 5/10; A01H 5/02

(52) U.S. Cl. CPC A01H 6/1416; A01H 5/02; A01H 5/10

(57) ABSTRACT

The invention relates to the Safflower variety designated SXT Bright Citrine. Provided by the invention are the seeds, plants and derivatives of the Safflower variety SXT Bright Citrine. Also provided by the invention are tissue cultures of the Safflower variety SXT Bright Citrine and the plants regenerated therefrom. Still further provided by the invention are methods for producing Safflower plants by crossing the Safflower variety SXT Bright Citrine with itself or another Safflower variety and plants produced by such methods.

### FIELD OF THE INVENTION

The present invention relates to the field of plant breeding. In particular, the invention provides safflower plants and seeds with high levels of pigment. The invention further provides for a new and distinct Safflower variety Sensient Bright Citrine and for breeding methods with these Safflower plants.

### BACKGROUND OF THE INVENTION

Safflower (Carthamus tinctorius) is a member of the compositae family. The safflower plant is a thistle-like annual with many branches, each branch having a flowerhead of bright yellow, orange or red flowers. Safflower was first cultivated in the Near East thousands of years ago. Traditionally, safflower was grown for its flowers, for use in dyes and in flavoring foods. More recently, safflower is grown for its seeds, as a source of edible oils and for use as birdseed.

So far, breeding efforts have provided a number of useful Safflower lines with beneficial traits, however, there remains a great need in the art for new lines with further improved traits. Thus, there is a need for new Safflower varieties with improved traits, particularly Safflower varieties with high pigment levels.

### SUMMARY OF THE INVENTION

The objective of the invention was to develop a Safflower variety with a high pigment content.

In one aspect the present invention provides seed of a Safflower variety, designated SXT Bright Citrine, having been deposited under Accession Number ______, a plant, or a part thereof, produced by growing said seed. The invention also provides methods and compositions relating to plants and plant parts, such as pollen, flowers, seeds, pods, leaves, stems and progenies of Safflower variety SXT Bright Citrine.

In another aspect, the invention provides a composition comprising a seed of Safflower variety SXT Bright Citrine comprised in plant seed growth media. In certain embodiments, the plant seed growth media is a soil or synthetic cultivation medium. In specific embodiments, the growth medium maybe comprised in a container or may, for example, be soil in a field. Plant seed growth media are well known to those of skill in the art and include, but are in no way limited to, soil or synthetic cultivation medium.

In a further aspect of the invention relates to a tissue culture of regenerable cells of the Safflower variety SXT Bright Citrine, as well as plants regenerated there from, wherein the regenerated Safflower plant is capable of expressing all the morphological and physiological characteristics of a plant grown from the Safflower seed designated SXT Bright Citrine.

Still yet another aspect of the invention relates to a first generation (F₁) hybrid Safflower seed produced by crossing a plant of the Safflower variety SXT Bright Citrine to a second Safflower plant. Also included in the invention are the F₁ hybrid Safflower plants grown from the hybrid seed produced by crossing the Safflower variety SXT Bright Citrine to a second Safflower plant.

Still yet another aspect of the invention is a method of producing Safflower seeds comprising crossing a plant of the Safflower variety SXT Bright Citrine to any second Safflower plant, including itself or another plant of the variety SXT Bright Citrine. In particular embodiments of the invention, the method of crossing comprises the steps of a) planting seeds of the Safflower variety SXT Bright Citrine; b) cultivating Safflower plants resulting from said seeds until said plants bear flowers; c) allowing fertilization of the flowers of said plants; and d) harvesting seeds produced from said plants.

Still yet another aspect of the invention is a method of producing hybrid Safflower seeds comprising crossing the Safflower variety SXT Bright Citrine to a second, distinct Safflower plant that is non-isogenic to the Safflower variety SXT Bright Citrine. In particular embodiments of the invention, the crossing comprises the steps of a) planting seeds of Safflower variety SXT Bright Citrine and a second, distinct Safflower plant, b) cultivating the Safflower plants grown from the seeds until the plants bear flowers; c) cross-pollinating a flower on one of the two plants with the pollen of the other plant; and d) harvesting the seeds resulting from the cross-pollinating.

Still yet another aspect of the invention is a method for developing a Safflower plant in a Safflower breeding program comprising: obtaining a Safflower plant, or its parts, of the variety SXT Bright Citrine; and b) employing said plant or parts as a source of breeding material using plant breeding techniques. In the method, the plant breeding techniques may be selected from the group consisting of recurrent selection, mass selection, bulk selection, backcrossing, pedigree breeding, and genetic marker-assisted selection. In certain embodiments of the invention, the Safflower plant of variety SXT Bright Citrine is used as the male or female parent.

Still yet another aspect of the invention is a method of producing a Safflower plant derived from the Safflower variety SXT Bright Citrine, the method comprising the steps of: (a) crossing a plant of the Safflower variety SXT Bright Citrine with a second Safflower plant to produce a progeny plant that is derived from Safflower variety SXT Bright Citrine; and (b) crossing the progeny plant with itself or a second plant to produce a progeny plant of a subsequent generation that is derived from a plant of the Safflower variety SKI Bright Citrine. In one embodiment of the invention, the method further comprises: (c) crossing the progeny plant of a subsequent generation with itself or a second plant to produce a progeny plant of a further subsequent generation that is derived from a plant of the Safflower variety SKI' Bright Citrine; and (d) repeating step (c), in some embodiments, at least 2, 3, 4 or more additional generations to produce an inbred Safflower plant that is derived from the Safflower variety SXT Bright Citrine. The invention still further provides a Safflower plant produced by this and the foregoing methods.

In another embodiment of the invention, the method of producing a Safflower plant derived from the Safflower variety SXT Bright Citrine further comprises: (a) crossing the Safflower variety SXT Bright Citrine derived Safflower plant with itself or another Safflower plant to yield additional Safflower variety SXT Bright Citrine derived progeny Safflower seed; (b) growing the progeny Safflower seed of step (a) under plant growth conditions to yield additional Safflower variety SXT Bright Citrine derived Safflower plants; and (c) repeating the crossing and growing steps of(a) and (b) to generate further Safflower variety SXT Bright Citrine derived Safflower plants. In specific embodiments, steps (a)and (b) may be repeated at least 1, 2, 3, 4, or 5 or more times as desired. The invention still further provides a Safflower plant produced by this and the foregoing methods.

### DETAILED DESCRIPTION OF THE DRAWINGS

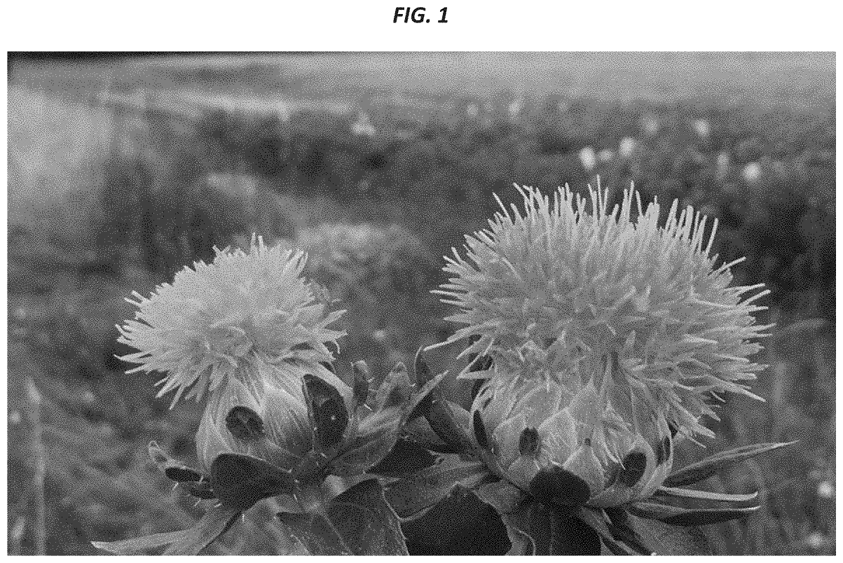

FIG. 1 shows a color photograph of SXT Bright Citrine.

### DETAILED DESCRIPTION OF THE INVENTION

The invention provides methods and composition relating to plants, seeds, and derivatives of the Safflower variety SXT Bright Citrine.

One aspect of the current invention concerns methods for crossing the Safflower variety SXT Bright Citrine with itself or a second plant and the seeds and plants produced by such methods. These methods can be used for propagation of the Safflower variety SXT Bright Citrine or can be used to produce hybrid Safflower seeds and the plants grown there from. Hybrid Safflower plants can be used by breeders in the commercial production of Safflower products or may be advanced in certain breeding protocols for the production of novel Safflower varieties. A hybrid plant can also be used as a recurrent parent at any given stage in a backcrossing protocol during the production of a single locus conversion of the Safflower variety SXT Bright Citrine. Safflower variety SXT Bright Citrine is well suited to the development of new varieties based on the elite nature of the genetic background of the variety. In selecting a second plant to cross with SXT Bright Citrine for the purpose of developing novel Safflower varieties, it will typically be desired to choose those plants that either themselves exhibit one or more selected characteristics or that exhibit the characteristic(s) when in hybrid combination. Examples of potentially selected characteristics include increased pigment content, increased flower size, multiple petals, broad environmental adaptation, and insect and pest resistance, and resistance to bacterial, fungal, or viral disease.

Choice of breeding or selection methods depends on the mode of plant reproduction, the heritability of the trait(s) being improved, and the type of variety used commercially (e.g., F₁ hybrid variety, pure line variety, etc.). For highly heritable traits, a choice of superior individual plants evaluated at a single location will be effective; whereas, for traits with low heritability, selection should be based on mean values obtained from replicated evaluations of families of related plants. Popular selection methods commonly include pedigree selection, modified pedigree selection, mass selection, recurrent selection and backcrossing.

The complexity of inheritance influences the choice of the breeding method, Backcross breeding is used to transfer one or a few genes for a highly heritable trait into a desirable variety. This approach has been used extensively (Bowers et al., Crop Sci., 32(1):67-72, 1992; Nickell and Bernard, Crop Sci., 32(3):835, 1992). Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross. Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful varieties produced per unit of input, e.g., per year, per dollar expended, etc.

Promising advanced breeding lines are thoroughly tested and compared to appropriate standards in environments that are representative of the commercial target area(s) for generally three or more years. The best lines are candidates for new commercial varieties. Those still deficient in a few traits may be used as parents to produce new populations for further selection.

These processes, which lead to the final step of marketing and distribution, may take as much as 8 to 12 years from the time the first cross is made. Therefore, development of new varieties is a time-consuming process that requires precise forward planning, efficient resource utilization, and minimal direction changes,

Identifying individuals that are genetically superior is a difficult task because the true genotypic value for most traits can be masked by other confounding traits or environmental factors. One method of identifying a superior plant is observing its performance relative to other experimental plants and one or more widely grown standard varieties. Single observations are generally inconclusive, while replicated observations provide a better estimate of genetic worth.

The goal of plant breeding is to develop new, unique, and superior Safflower varieties and hybrids. The breeder initially selects and crosses two or more parental lines. This is generally followed by repeated selling and selection, which produces many new genetic combinations. Each year, the plant breeder selects the germplasm to advance to the next generation. This germplasm is grown under unique and different geographical, climatic, and soil conditions, and further selections are then made during and at the end of the growing season. The varieties which are developed are unpredictable. This unpredictability is because the breeder's selection occurs in unique environments, with no control at the DNA level (using conventional breeding procedures), and with millions of different possible genetic combinations being generated. A breeder of ordinary skill in the art cannot predict the final resulting lines he develops, except possibly in a gross and general fashion. The same breeder cannot produce the same variety twice by using the exact same original parents and the same selection techniques. This unpredictability results in the expenditure of large amounts of research monies to develop superior new Safflower varieties.

Pedigree breeding and recurrent selection breeding methods are used to develop varieties from breeding populations. Breeding programs combine traits from two or more varieties or various broad-based sources into breeding pools from which varieties are developed by selfing and selection of phenotypes. The new varieties are evaluated to determine which have commercial potential.

Pedigree breeding is commonly used for the improvement of self-pollinating crops. Two parents which possess favorable, complementary traits are crossed to produce F₁ progeny. An F₂ population is then produced by selfing one or several F₁ plants. Selection of the best individuals may begin in the F₂ population or later depending upon the breeder's objectives then, beginning in the F₃ generation, the best individuals in the best families can be selected. Replicated testing of families can begin in the F₃ or generations to improve the effectiveness of selection for traits of low heritability. At an advanced stage of inbreeding (i.e., the F₆ and F₇ generations), the best lines or mixtures of phenotypically similar lines are tested for potential release as new varieties.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population from which further cycles of selection are continued.

Backcross breeding has been used to transfer genetic loci for simply inherited or highly heritable traits into a homozygous variety that is used as the recurrent parent. The source of the trait to be transferred is called the donor or non-recurrent parent. The resulting plant is expected to have the attributes of the recurrent parent and the trait transferred from the donor parent. After the initial cross, individuals possessing the phenotype of the donor parent are selected and repeatedly crossed, i.e., backcrossed, to the recurrent parent. The resulting plant is expected to have the attributes of the recurrent parent (i.e., variety) and the desirable trait transferred from the donor parent. The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which the lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed. Descriptions of other breeding methods that are commonly used for different traits and crops can be found in one of several reference books (e.g., Allard. “Principles of Plant Breeding,” John Wiley & Sons, NY, University of California, Davis, California, 50-98, 1960; Simmonds. “Principles of Crop Improvement,” Longman, Inc., NY, 369-399, 1979; Sneep et al., “Plant Breeding Perspectives,” Wageningen (ed.), Centre for Agricultural Publishing and Documentation, 1979.

Proper testing should detect any major faults and establish the level of superiority or improvement over current varieties, In addition to showing superior performance, there must be a demand for a new variety that is compatible with industry standards or which creates a new market. The introduction of a new variety will incur additional costs to the seed producer, the grower, processor, and consumer. The testing preceding release of a new variety should take into consideration research and development costs as well as the technical superiority of the final variety. For seed-propagated varieties, it must be feasible to produce seed easily and economically. In addition to phenotypic observations, a plant can also be identified by its genotype. The genotype of a plant can be characterized through a molecular marker profile, which can identify plants of the same variety or a related variety, can identify plants and plant parts which are genetically superior as a result of an event comprising a backcross conversion, which can be used to determine or validate a pedigree. Such molecular marker profiling can be accomplished using a variety of techniques including, but not limited to, restriction fragment length polymorphism (RFLP), amplified fragment length polymorphism (RFLP), sequence-tagged sites (STS), randomly amplified polymorphic DNA (RAPD), arbitrarily primed polymerase chain reaction (AP-PCR), DNA amplification fingerprinting (DAF), sequence characterized amplified regions (SCARs), variable number tandem repeat (VNTR), short tandem repeat (STR), single feature polymorphism (SFP), simple sequence length polymorphism (SSLP),restriction site associated DNA, allozymes, isozyme markers, single nucleotide polymorphisms (SNPs), or simple sequence repeat (SSR) markers, also known as microsatellites (Gupta et al., 1999; Korzun et at, 2001). Various types of these markers, for example, can be used to identify individual varieties developed from specific parent varieties, as well as cells or other plant parts thereof. For example, see Berry et al. (2003)“Assessing Probability of Ancestry Using Simple Sequence Repeat Profiles: Applications to Maize inbred Lines and Safflower Varieties” Genetics 165(1):331-342, each of which are incorporated by reference herein in their entirety.

In some examples, one or more markers may be used to characterize and/or evaluate a Safflower variety. Particular markers used for these purposes are not limited to any particular set of markers but are envisioned to include any type of marker and marker profile that provides a means for distinguishing varieties. One method of comparison may be to use only homozygous loci for Safflower variety SXT Bright Citrine. Primers and PCR protocols for assaying these and other markers, in addition to being used for identification of Safflower variety SXT Bright Citrine, as well as plant parts and plant cells of Safflower variety SXT Bright Citrine, a genetic profile may be used to identify a Safflower plant produced through the use of Safflower variety SXT Bright Citrine or to verify a pedigree for progeny plants produced through the use of Safflower variety SXT Bright Citrine. A genetic marker profile may also be useful inbreeding and developing backcross conversions.

In an embodiment, the present invention provides a Safflower plant characterized by physiological data obtained from a representative sample of said variety deposited with the American Type Culture Collection (ATCC). Thus, plants, seeds, or parts thereof, having all or essentially all of the morphological and physiological characteristics of Safflower variety SXT Bright Citrine are provided. Further provided is a Safflower plant formed by the combination of the disclosed Safflower plant or plant cell with another Safflower plant or cell and comprising the homozygous alleles of the variety.

In some examples, a plant, a plant part, or a seed of Safflower variety SXT Bright Citrine may be characterized by producing a molecular profile. A molecular profile may include, but is not limited to, one or more genotypic and/or phenotypic profile(s). A genotypic profile may include, but is not limited to, a marker profile, such as a genetic map, a linkage map, a trait maker profile, a SNP profile, an SSR profile, a genome-wide marker profile, a haplotype, and the like.

A molecular profile may also be a nucleic acid sequence profile, and/or a physical map. A phenotypic profile may include, but is not limited to, a protein expression profile, a metabolic profile, an mRNA expression profile, and the like. One means of performing genetic marker profiles is using SSR polymorphisms that are well known in the art. A marker system based on SSRs can be highly informative in linkage analysis relative to other marker systems, in that multiple alleles may be present. Another advantage of this type of marker is that through use of flanking primers, detection of SSRs can be achieved, for example, by using the polymerase chain reaction (PCR), thereby eliminating the need for labor-intensive Southern hybridization. PCR detection maybe performed using two oligonucleotide primers flanking the polymorphic segment of repetitive DNA to amplify the SSR region. Following amplification, markers can be scored by electrophoresis of the amplification products. Scoring of marker genotype is based on the size of the amplified fragment, which correlates to the number of base pairs of the fragment. While variation in the primer used or in the laboratory procedures can affect the reported fragment size, relative values should remain constant regardless of specific primer or laboratory used.

When comparing varieties, it may be beneficial to have all profiles performed in the same lab. A genotypic profile of Safflower variety SXT Bright Citrine can be used to identify a plant comprising Safflower variety SXT Bright Citrine as a parent, since such plants will comprise the same homozygous alleles as variety SXT Bright Citrine. Because the Safflower variety is essentially homozygous at all relevant loci, most loci should have only one type of allele present. In contrast, a genetic marker profile of an F₁ progeny should be the sum of those parents, e.g., if one parent was homozygous for allele X at a particular locus, and the other parent homozygous for allele Y at that locus, then the F₁ progeny will be XV (heterozygous) at that locus. Subsequent generations of progeny produced by selection and breeding are expected to be of genotype XX (homozygous), YY (homozygous), or XV (heterozygous) for that locus position. When the F₁ plant is selfed or sibbed for successive filial generations, the locus should be either X or Y for that position.

A genotypic profile of variety SXT Bright Citrine also can be used to identify essentially derived varieties and other progeny varieties developed from the use of variety SXT Bright Citrine, as well as cells and other plant parts thereof. Plants of the invention include any plant having at least 90%, 91%, 92%,93%, 94%, 95%, 96%, 97%, 98%, 99%, 99.5%, or 99.9% of the markers in the genotypic profile, and that retain 90%,91%, 92%, 93%, 94%, 95%, 96%, 97%, 98%, 99%, 99.5%, or 99.9% of the morphological and physiological characteristics of variety SXT Bright Citrine when grown under the same conditions. Such plants may be developed using markers well known in the art. Progeny plants and plant parts produced using variety SXT Bright Citrine may be identified, for example, by having a molecular marker profile of at least 25%, 30%, 35%, 40%, 45%, 50%, 55%, 60%, 65%, 70%,75%, 76%, 77%, 78%, 79%, 80%, 81%, 82%, 83%, 84%,85%, 86%, 87%, 88%, 89%, 90%. 91%, 92%, 93%, 94%,95%, 96%, 97%, 98%, 99%, or 99.5% genetic contribution from Safflower variety SXT Bright Citrine, as measured by either percent identity or percent similarity. Such progeny may be further characterized as being within a pedigree distance of variety SXT Bright Citrine, such as within 1, 2, 3, 4, or 5 or less crosspollinations to a Safflower plant other than variety SXT Bright Citrine, or a plant that has variety SXT Bright Citrine as a progenitor, Unique molecular profiles may be identified with other molecular tools, such as SNPs and RFLPs.

Any time the Safflower variety SXT Bright Citrine is crossed with another, different, variety, first generation (F₁) Safflower progeny are produced. The hybrid progeny are produced regardless of characteristics of the two varieties produced. As such, an F₁ hybrid Safflower plant may be produced by crossing SXT Bright Citrine with any second Safflower plant. The second Safflower plant may be genetically homogeneous (e.g., inbred) or may itself be a hybrid. Therefore, any F₁ hybrid Safflower plant produced by crossing Safflower variety SXT Bright Citrine with a second Safflower plant is a part of the present invention.

Further Embodiments of the Invention

In certain aspects of the invention, plants of Safflower variety SXT Bright Citrine are modified to include at least a first heritable trait. Such plants may, in one embodiment, be developed by a plant breeding technique called backerossing, wherein essentially all of the morphological and physiological characteristics of a variety are recovered in addition to a genetic locus transferred into the plant via the backcrossing technique. By essentially all of the morphological and physiological characteristics, it is meant that the characteristics of a plant are recovered that are otherwise present when compared in the same environment, other than occasional variant traits that might arise during backcrossing.

In a typical backcross protocol, the original variety of interest (recurrent parent) is crossed to a second variety (nonrecurrent parent) that carries the single locus of interest to be transferred. The resulting progeny from this cross are then crossed again to the recurrent parent and the process is repeated until a Safflower plant is obtained wherein essentially all of the morphological and physiological characteristics of the recurrent parent are recovered in the converted plant, in addition to the transferred locus from the nonrecurrent parent.

The selection of a suitable recurrent parent is an important step for a successful backcrossing procedure. The goal of a backcross protocol is to alter or substitute a trait or characteristic in the original variety. To accomplish this, a locus of the recurrent variety is modified or substituted with the desired locus from the nonrecurrent parent, while retaining essentially all of the rest of the genome of the original variety, and therefore the morphological and physiological constitution of the original variety. The choice of the particular nonrecurrent parent will depend on the purpose of the backcross; one of the major purposes is to add an agronomically important trait to the plant. The exact backcrossing protocol will depend on the characteristic or trait being altered to determine an appropriate testing protocol. Although backcrossing methods are simplified when the characteristic being transferred is a dominant allele, a recessive allele may also be transferred. In this instance, it may be necessary to introduce a test of the progeny to determine if the desired characteristic has been successfully transferred.

Many traits have been identified that are not regularly selected for in the development of a new inbred but that can be improved by backcrossing techniques. These traits include, but are not limited to, increased pigment content, increased flower size, multiple petals, broad environmental adaptation, and insect and pest resistance, and resistance to bacterial, fungal, or viral disease. These comprise genes generally inherited through the nucleus.

Selection of Safflower plants for breeding is not necessarily dependent on the phenotype of a plant and instead can be based on genetic investigations. For example, one may utilize a suitable genetic marker that is closely associated with a trait of interest. One of these markers may therefore be used to identify the presence or absence of a trait in the offspring of a particular cross, and hence may be used in selection of progeny for continued breeding. This technique may commonly be referred to as marker assisted selection. Any other type of genetic marker or other assay that is able to identify the relative presence or absence of a trait of interest in a plant may also be useful for breeding purposes. Procedures for marker assisted selection applicable to the breeding of Safflower are well known in the art. Such methods will be of particular utility in the case of recessive traits and variable phenotypes, or when conventional assays may be more expensive, time consuming or otherwise disadvantageous. Genetic markers that could be used in accordance with the invention include, but are not necessarily limited to, Simple Sequence Length Polymorphisms (SSLPs) (Williams et al., Nucleic Acids Res., 18:65316535,1990), Randomly Amplified Polymorphic DNAs (RAPDs), DNA Amplification Fingerprinting (DAF), Sequence Characterized Amplified Regions (SCARs), Arbitrary Primed Polymerase Chain Reaction (AP-PCR), Amplified Fragment Length Polymorphisms (AFLPs) (European Patent Application Publication No. EP0534858, incorporated herein by reference in its entirety), and Single Nucleotide Polymorphisms (SNPs) (Wang et al., Science, 280:1077-1082, 1998).

Carthamus Red
A. Synoinvris—Safflower red, carthamic acid, CI Natural Red 26 (No. 75140)
B. Definition—Carthamus Red, a flavonvoid, is obtained from the dried petals of Carthamus tinctorius L. To obtain carthamus red, carthamus yellow is extracted from the petals with water and the residue treated with aqueous sodium hydroxide or other alkali. Carthamus red is precipitated from the extract by addition of acid, separated by filtration and dried. The principal coloring matter is carthamin. Food grade materials such as dextrin may be added as carriers for manufacturing dry, powdered items of commerce.
C. Chemical formula C₄₃H₄₂O₂₂ (carthamin)

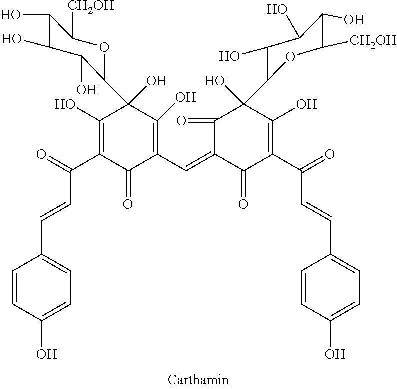 [SMILES: O=C(/C=C/c1ccc(O)cc1)C1=C(O)C(O)(C2OC(CO)C(O)C(O)C2O)C(=O)/C(=C\C2=C(O)C(O)(C3OC(CO)C(O)C(O)C3O)C(O)=C(C(=O)/C=C/c3ccc(O)cc3)C2=O)C1=O]

D. Formula Weight—910.81 (carthamin)
E. Description—Dark red to red-brown powder with a characteristic slight odor
F. Functional Uses Color
G. Characteristics
   a. Very slightly soluble in water and in ethanol; practically insoluble in ether
   b. A solution of the sample in dimethyl formamide is red and shows an absorption maximum between 525-535 nm.
H. Identification Tests
a. Thin layer chromatography Activate some silica gel for 1 h at 110° and prepare a TLC plate. Prepare an 0.02% solution of the sample in dimethylformamide and apply 20 ul to the plate. Allow to dry and develop using a mixture of n-butanol, acetic acid and water (4:1:2 by volume) until the solvent front has ascended about 10 cm. Allow to dry. Carthamin appears as a red spot with an R_{f} value of about 0.40.
   b. Color reactions—Dissolve 10 mg of the sample in 50 ml water. The color of the solution is red. Add alkali to raise the pH to above 7. The color changes to orange-yellow. To 0.05 g of the sample add 2 ml of 5% phosphoric acid and heat for 1 h on a water bath. After cooling, filter and wash the residue with 3 ml of water. Combine the filtrate and the washings. Neutralize the combined solution with sodium hydroxide TS, add 5 ml of Fehling's TS and heat on a water bath for 10 min. A red precipitate is produced.
I. Punk Tests
   a. Synthetic dyes—Basic dyes: To 1 g of the sample add 100 ml of 1% sodium hydroxide solution, and mix well. Extract 30 ml of this solution with 15 ml of ether. Then extract the ether layer twice with dilute acetic acid (5 ml); the dilute acetic acid layer does not contain any color.
   b. Acidic dyes—To 1 g of the sample add 1 ml of ammonia TS and 8 ml of water, and shake well. Discard an oily layer when separated. Proceed as directed in Paper Chromatography (Ascending Chromatography) using 20 μl of the solution as the sample solution, and a mixture of pyridine and ammonia TS (2:1 by volume) as the developing solvent. Stop the development when the solvent front has advanced about 15 cm from the point of application. No spot is observed at the solvent front after drying under daylight. If any spot is observed, it should be decolorized when sprayed with a solution of stannous chloride in hydrochloric acid (2 in 5).
J. Method of Assay—Transfer about 0.01 g of the sample, accurately weighed, in a 300-ml ground stoppered flask, add 150 ml of dimethylformamide (DMFA), dissolve by shaking occasionally and allow stand for 2 hours. Filter this solution through a glass filter into a 200-ml volumetric flask. Wash the flask and filter with two 25-ml portions of DMFA, combine the filtrate and the washings, add DMFA to volume and mix. Dilute if necessary. Determine the absorbance (A) at the maximum absorbance in the range of 525-535 nm using a 1-cm cell with DMFA as a blank and calculate the percent of coloring matter (P) with the following formula/taking any additional dilution into account:

$$\underline{P = ( A / 992 ) \times ( 200 / W ) , where W is weight of sample in g .}$$

$$\underline{P = \frac{A}{992} \times \frac{200}{W}}$$

### EXAMPLES

### Example 1

### Development of Safflower Variety SXT Bright Citrine

The Safflower variety SXT Bright Citrine was developed from a plant selection program and line purification trials. The selection program started in year 1 to identify single plants for average flowering time, average plant height (cm), average single flowers per plant (cm), average size of the single flower heads, and homogeneity of flowering. In year 2, 38 single plants were selected for re-growing. In year 2, 13 single plants were selected for re-growing. In year 3, 5 plants were selected for re-growing. In 2019, the 5 plants from year 3 were re-grown in repetition (2×), with a target selection of 1 to 2 elite Safflower varieties.

TABLE 1
Safflower plant selection.

| | | | | Average | Average | |
|---|---|---|---|---|---|---|
| | | | Average | Single | Size of | |
| | | Average | Plant | Flowers | the Single | |
| | | Flowering | Height | per Plant | Flower | Homogeneity |
| Code | Line | Time | (cm) | (cm) | Heads | of Flowering |
| 1 | 17-02-WH1, E2 | Medium | 82.2 | 13.5 | 9 | 7 |
| 2 | 17-17-WH2, E1 | Medium | 92.5 | 15.2 | 9 | 7 |
| 3 | 17-18-WH2, E1 | Late | 91.7 | 14.6 | 9 | 9 |
| 4 | 17-33-WH2, E1 | Early | 92.6 | 17.5 | 9 | 7 |
| 5 | 17-35-WH2, E1 | Late | 83.3 | 14.6 | 9 | 7 |
| 6 | 17-02-WH1, E2 | Medium | 92.3 | 14.8 | 9 | 7 |
| 7 | 17-17-WH2, E2 | Medium | 91.3 | 12.3 | 9 | 7 |
| 8 | 17-18-WH2, E2 | Late | 94.8 | 17.6 | 9 | 7 |
| 9 | 17-33-WH2, E2 | Early | 72.7 | 15.6 | 9 | 9 |
| 10 | 17-35-WH2, E2 | Late | 84.8 | 16.4 | 9 | 7 |

1. A plant of Safflower variety SXT Bright Citrine, wherein representative seed of Safflower variety have been deposited under ATCC Accession No ______.

2. A plant part of the plant of claim 1, wherein the plant part comprises at least one cell of the plant.

3. A seed of Safflower variety SXT Bright Citrine, wherein representative seed of Safflower variety have been deposited under ATCC Accession No ______.

4. A method of producing Safflower seed, the method comprising crossing the plant of claim 1 with itself or a second Safflower plant to produce Safflower seed.

5. The method of claim 4, the method further comprising crossing the plant of Safflower variety SXT Bright Citrine with a second, non-isogenic Safflower plant to produce Safflower seed.

6. An F₁ Safflower seed produced by the method of claim 5.

7. A Safflower plant produced by growing the F₁ Safflower seed of claim 6.

8. A composition comprising the seed of claim 3 comprised in plant seed growth media.

9. The composition of claim 8, wherein the plant seed growth media is soil or a synthetic cultivation medium.

10. The plant Safflower variety SXT Bright Citrine of claim 1, comprising a single locus conversion, wherein the plant otherwise comprises all of the morphological and physiological characteristics of Safflower variety SXT Bright Citrine when grown under the same environmental conditions, and wherein representative seed of Safflower variety SXT Bright Citrine have been deposited under ATCC Accession No ______.

11. A seed that produces the plant of claim 10.

12. The seed of claim 11, wherein the single locus confers a trait selected from increased pigment content, increased flower size, multiple petals, broad environmental adaptation, and insect and pest resistance, and resistance to bacterial, fungal, or viral disease.

13. The method of claim 5, the method comprising: crossing a plant grown from Safflower seed with itself or a different Safflower plant to produce seed of a progeny plant of a subsequent generation;
a. growing a progeny plant of a subsequent generation from seed of a progeny plant of a subsequent generation and crossing the progeny plant of a subsequent generation with itself or a second plant to produce seed of a progeny plant of a further subsequent generation; and
b. repeating step (a) with sufficient inbreeding to produce seed of an inbred Safflower plant that is derived from Safflower variety SXT Bright Citrine.

14. The method of claim 13, the method comprising crossing a plant grown from seed of an inbred Safflower plant that is derived from Safflower variety SXT Bright Citrine with a non-isogenic plant to produce seed of a hybrid Safflower plant that is derived from Safflower variety SXT Bright Citrine.

15. A method of producing a commodity plant product, the method comprising producing the commodity plant product from the plant of claim 1.

16. The method of claim 15, wherein the commodity plant product is a pigment.
